(19) United States
(12) Patent Application Publication
(10) Pub. No.: US 2022/0195414 A1
(43) Pub. Date: Jun. 23, 2022

(54) Improved Manufacturing Procedures for Cell Based Therapies

(71) Applicant: Russell Biotech, Inc., Doylestown, PA (US)

(72) Inventor: Thomas Russell, Doylestown, PA (US)

(73) Assignee: Russell Biotech, Inc., Doylestown, PA (US)

(21) Appl. No.: 17/600,432

(22) PCT Filed: Mar. 24, 2020
(86) PCT No.: PCT/US2020/024456

§ 371 Date: Sep. 30, 2021

Related U.S. Application Data
(60) Provisional application No. 62/830,676, filed on Apr. 8, 2019.

(51) Int. Cl. C12N 13/00

(52) U.S. Cl. CPC C12N 13/00

(57) ABSTRACT

CAR T cell therapies have shown promise in treating human blood cell cancer. The preparation of CAR T cells involves many complex, time consuming steps prior to infusion of the CAR T cells into a cancer patient. One step in the process to create CAR T cells often involves using magnetic separation technologies to isolate specific subsets of T cells prior to creating the CAR T cells. When using current magnetic separation technologies to remove undesired cell populations the recovery of the desired cell population can be as low as 50-70% or even lower and the procedures often take 30-60 minutes. In the case of autologous CAR T cell therapies such cell loss is often not acceptable. The present invention offers means to improve the recovery of desired cells to close to 100% very rapidly thus significantly improving a step in the manufacture of CART cells and in many cases will make such therapy possible for a larger patient population.

### CROSS REFERENCE TO RELATED APPLICATIONS

This application is the US national application of PCT/US2020/024456, filed 24 Mar. 2020, and which claims benefit of and priority to U.S. Provisional Patent Application No. 62/830,676, filed 8 Apr. 2019, now expired, the disclosure of which is incorporated by reference in its entirety.

### BACKGROUND

### 1. Field of the Invention

The invention provides improved manufacturing procedures, using dense, metallic magnetic micro/nano particles, for the preparation of cell therapies for the treatment of cancer. More specifically the invention relates to an improved cell separation procedure that yields much higher numbers of desired cells than that of existing magnetic particle separation technology. The invention also includes kits and reagents for manufacturing the desired enriched cell populations.

### 2. Description of Related Art

Over the past decade, a patient's own immune response has become established as the fourth therapeutic option in the treatment of hematological malignancies, along with surgery, chemotherapy and radiotherapy. In particular, the adoptive transfer of a patient's T cells, redirected toward the cancer cells with a chimeric antigen receptor (CAR), has achieved spectacular remissions in B cell malignancies (leukemia/lymphoma) and is beginning to show promise for solid cancers (21). The CAR is an artificial receptor molecule with an extracellular cancer-targeting module and an intracellular T cell-activating module (2).

CAR T cell therapy involves a number of distinct steps that are needed in order to produce the cell-based therapy (4):

1. Isolation of PBMCs usually via leukapheresis is routinely used to obtain T cells and T cell subsets. Currently DYNAL magnetic particles and Miltenyi magnetic particles are used for this step usually in a negative selection procedure to deplete undesired cells but positive selection can also be used but often requires removal of the magnetic particles before proceeding.

In the future if technology becomes available it is desired to use whole blood rather than pheresis material so that the patient does not have to go through leukapheresis. Also, the initiation of manufacturing procedures with defined subpopulations of T cells that can be derived from a blood draw instead of a leukapheresis product would reduce the scale and therefore the cost of manufacturing CAR T cells for therapy (4).

2. Isolated T cells or T cell subsets are then activated using CD3/CD28 particles.

3. The desired cell population(s) are transduced usually with a virus to create the CAR T cell i.e. CAR T cells that will eliminate B cell malignancies expressing CD19.

4. CAR T cells are expanded further.

5. CAR T cells are cryopreserved.

6. CAR T cells are then thawed and infused into the patient.

According to clinical trials.gov as of November 2017, there are currently more than 260 active, recruiting or completed worldwide trials utilizing CAR T cells; 92 trials utilizing T cell receptor modified T Cells (CAR T cells) for treatment of various hematological cancers, which hold promise to potentially cure where current standard treatments of chemotherapy, radiation therapy and surgery prove unsuccessful.

The field has advanced based primarily on use of autologous transplantation but arguments have been made that allogeneic transplantation may be the way to go in the future simply because universal donors would be available and it would obviate the need to use autologous cells from a sick patient. As part of the description of the related art both autologous and allogeneic CAR T cell therapies will be discussed as the invention disclosed here will impact both CAR T cell therapeutic approaches.

Autologous CAR T Cell Therapy:

In autologous immunotherapies, the starting material is typically collected through leukapheresis, where the leukocytes are separated out and the remaining blood products are returned to the patient. As there is inherent variability in the cell populations in these leukapheresis products, processes to remove unwanted cells or isolate specific populations of cells have been developed using a variety of technologies including physical separation via centrifugation, magnetic, fluorescent, as well as acoustic based selection (3). Antibodies can be used to isolate cells based on cell surface marker expression. These antibodies can be conjugated to fluorochromes or magnetic beads.

In CAR T cells, tumor recognition is mediated by a single chain variable fragment (scFv) derived from a monoclonal antibody or an antigen-binding region isolated from an immunoglobulin heavy and light chain library. Unlike T cell receptor (TCR) mediated antigen recognition, CAR T cells function independently of HLA and can therefore be used in any genetic background. Second generation CAR T cells not only mediate antigen recognition and initiate T cell activation but also harness co-stimulation to enhance T cell function and prolong T cell persistence.

Though autologous CAR T cell therapy began using mononuclear cells derived from leukapheresis materials as starting cellular material it has become apparent early on that some fractionization of the material may be necessary before developing CAR T cells:

1. The inadvertent genetic engineering of a single leukemic cell during clinical manufacturing of a CAR-T cancer therapy led to fatal treatment resistance in one patient highlighting the margin for error in producing the cutting edge medicines. Researchers traced the relapse to the unintentional introduction of a leukemic B cell during the production of the personalized CAR T cell therapy. These findings illustrate the need for improved manufacturing technologies that can purge residual contaminating tumor cells from engineered T cells (6,16) preferably from the leukapheresis material before expansion but may need to be repeated prior to infusion into the patient.

2. Controlling the cellular composition of a CART cell product has the potential to reduce product variability, improve the consistency of in vivo proliferation and provide reproducible potency. Early investigational CAR T cell products used pooled T cells derived from unfractionated PBMCs as their source material. This can lead to inconsistent results. Besides purging any residual tumor cells it is becoming clear that CD4 and CD8 lymphocyte subsets may be more desirable than unfractionated PBMCs obtained by leukapheresis.

CAR-T cells generated from CD3+ population are widely used in clinical trials. However, studies from different laboratories have demonstrated that certain sub-sets of T cells such as naïve, central memory stem cells may display functional advantages.

The preferred procedure to date for T cell subset isolation has been to purge contaminating tumor cells and to isolate specific CD4 and CD8 lymphocyte subsets using magnetic bead-based separation technologies including those provided by Miltenyi (U.S. Pat. No. 5,411,863) and Thermo Fisher (DYNAL; U.S. Pat. No. 4,654,267) but not limited thereto. The particles are composed of inorganic metal oxide particles 30 nm or less in diameter imbedded in non-magnetic polymer material as described in U.S. Pat. No. 5,411,863 (Miltenyi); U.S. Pat. No. 4,654,267 (Ugelstad). Magnetic particles available commercially are by and large made of these inorganic iron oxides. But these magnetic particles of the art are less than desirable for CAR T cell therapy since they yield a desired cell population in unacceptable yield. When purging a cell population using these technologies reported recoveries of desired cells range from 26% (U.S. Pat. No. 10,081,793) to 60-70% (BD Biosciences Catalog). Another example: A problem exists with this technology as shown in reference (18): it includes sequential 2-step CliniMACS procedure for negative selection. After two CiniMACS procedures, CD8Tcm cell recovery was 26%.

Such cell recoveries are not acceptable for the manufacture of CAR T cells for cell therapy especially when using patients PBMCs. There is a definite need for an improved magnetic separation procedure that results in higher recovery of the starting cell material prior to developing the CAR T cells. These magnetic separation technologies based on inorganic iron oxides do not work well in undiluted whole blood and are time consuming.

The initiation of manufacturing procedures with defined subpopulations of T cells that can be derived from a blood draw instead of a leukapheresis product would reduce the scale and therefore the cost of manufacturing CAR T cells for therapy (4). The current magnetic separation technologies that are being used in the manufacture of CAR T cells do not work easily in undiluted whole blood. The existing magnetic particle technology often requires long times to obtain the desired T cell subsets.

Allogeneic CAR T Cell Therapy:

Adoptive T-cell therapy is now widely recognized as a breakthrough technology with the potential to become a curative option for certain types of cancers. However, persistent scaling challenges remain based on the highly personalized nature of the therapy. There is a definite desire to use “off the shelf” CAR T cells produced from allogeneic donors to circumvent the issues seen with autologous CAR T cells.

The availability of “off-the-shelf” or allogeneic T cells from an outside donor vastly decreases the current roadblocks of this technology. The allogeneic cells are engineered to bypass the barriers of the patient's immune system. Briefly, the features engineered into the cells include mechanisms to avoid immune reaction due to their non-matched HLA locus, as well as to ensure they are not replaced by the patient's own T cells over time. The majority of the patient's normal immune cells were removed prior to transplant of the ‘off’-the-shelf CAR T cells, minimizing the chance of rejection.

Despite the potential usefulness as a cancer treatment, allogeneic CAR T cell therapy has been limited, in part, by expression of the endogenous T cell receptor on the cell surface. CAR T cells expressing an endogenous T cell receptor may recognize major and minor histocompatibility antigens following administration to an allogenic patient, which can lead to the development of graft-versus-host-disease (GVHD).

Thus, it would be advantageous to develop allogeneic CAR T cells, prepared using T cells from a third-party donor, that have reduced expression of the endogenous T cell receptor and thus do not initiate GVHD upon administration.

A number of researches at universities and companies are working on providing a genetically-modified cell derived from a human T cell. Such a cell has a reduced cell-surface expression of the endogenous TCR when compared to an unmodified control cell (U.S. Pat. No. 10,093,900). Such a CAR T cell would be expected to be an effective cancer therapy and avoid destructive GVHD.

So-called universal T cells, which can be applied to a number of patients independently of their major histocompatibility complex, would make a priori production of CART Cells possible.

To the extent that these allogenic CAR T cells are manufactured in a manner similar to autologous CAR T cells it is clear that magnetic particles as described above and available commercially from a number of vendors would also be used in the preparation of allogeneic CAR T cells. As in autologous CAR T cell therapies, these existing magnetic particle technologies have significant disadvantages as described above including ease of use, reaction kinetics and significant loss of desired cell populations following negative selection/purging to remove undesired cells.

There is a definite need to improve the manufacturing procedures used for both autologous and allogeneic CAR T cell manufacture. Specifically, an improved magnetic particle separation procedure that will work in pheresis material, whole blood and PBMCs is needed that yields the desired cell populations in close to 100% recovery thus enabling the expansion of CART cells, but not limited to, using less leukapheresis material or whole blood. The invention described herein solves this problem providing a means to isolate undesired cell populations, rapidly and in high yield and to ensure the correct cells are expanded to develop the desired cell therapies whether autologous or allogeneic.

### SUMMARY OF THE INVENTION

The invention disclosed herein provides an improved magnetic particle/procedure that overcomes the problem seen with current magnetic particles: significant losses of desired cells following a purging step to remove undesired cells. In a preferred embodiment of the invention dense/magnetic nickel particles are used to remove undesired cell populations using multiple rounds of depletion if needed but yielding a desired cell population in close to 100% yield. The invention is well suited for manufacturing procedures related to cell therapies such as CAR T cell therapies but not limited thereto. Cell therapies such as CAR T cell therapies require a number of steps in the preparation of the CAR T cells that are used to treat a cancer patient. As a result of multiple steps involved it is inevitable that cell losses will occur at each step. For steps that require depletion of cells using magnetic particles, the particles of the invention disclosed here do not result in the loss of desired cells thus leading to a significant improvement over existing technology. An improvement that will further enable the improved manufacture of cell-based therapies used to treat malignancies such as leukemias/lymphomas and solid tumors. The particles of the invention are composed of solid metal. As a result of this sterilization of the particles, an absolute requirement for a therapeutic application, is straight forward compared to magnetic particles of the art composed of iron oxides. The particles, prior to the addition of reactants to the metal surface, are simply heated to 250 degrees centigrade for the appropriate time to sterilize the particles.

### BRIEF DESCRIPTIONS OF DRAWINGS

FIG. 1. Particles of the invention were used to demonstrate a key feature of the disclosed invention: that depletion/purging of an undesired cell population results in almost quantitative recovery of non-depleted cells. In this experiment 3.5 micron particles bound with the reactant mouse-anti-human CD8 were used to perform 6 rounds of purging resulting in no significant loss of the desired CD4 cells.

FIG. 2. The table demonstrates improved recoveries of non-targeted, desired cells compared to the competition

FIG. 3. The table demonstrates properties of the particles of the invention that enable them to provide unique performance characteristics. These unique performance characteristics enable the use of the technology to significantly improve sample preparation/manufacture for cell-based therapies including, but not limited to, CAR T cell therapy. These unique performance characteristics include a solid ferromagnetic core and a density close to 9 g/cc.

FIG. 4. Demonstrates the end-over-end mixing that enables binding of particles of the invention to target cells

FIG. 5. Demonstrates removal of specific cell populations using magnetic particles of the invention while leaving non-targeted cells in extremely high recovery. The top figure represents PBMCs; bottom box: lymphocytes; middle box: monocytes; top box granulocytes. The sample was run on a BD Flow Cytometer. Parameters measured: forward light scatter and 90 degree light scatter. The middle figure shows the results after the sample was treated with CD15+ magnetic particles of the invention. The bottom figure shows depletion of all leukocytes (lymphocytes, monocytes and granulocytes) with a combination of separate magnetic particles of the invention bound with anti-CD45, anti-CD4 and anti-CD15 monoclonal antibodies. The small depletion of monocytes (17.5%; middle figure) is specific depletion as it is known in the art that a subset of monocytes have CD15 on their surface.

### DETAILED DESCRIPTION OF PREFERRED EMBODIMENTS

A preferred embodiment of the disclosed invention to improve the manufacture of cell-based therapies for the treatment of cancer uses magnetic, dense nickel particles bound to reactants that recognize a cell surface antigen present on a cell population that is to be removed prior to making the cellular therapy product. The particle is described in U.S. Pat. No. 9,435,799 (799) and is incorporated herein by reference.

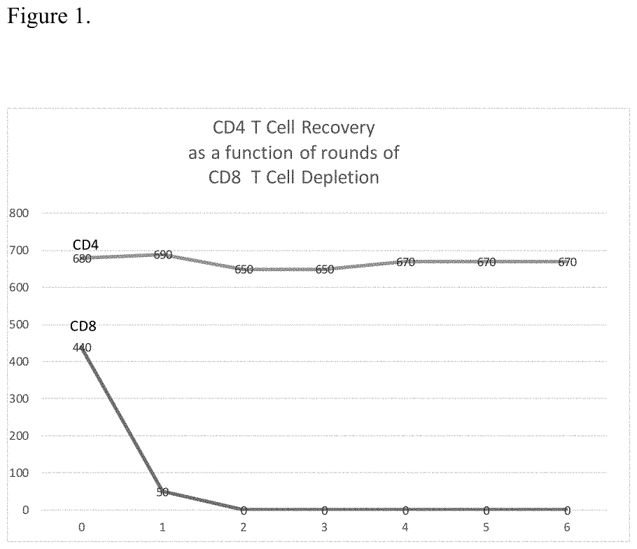

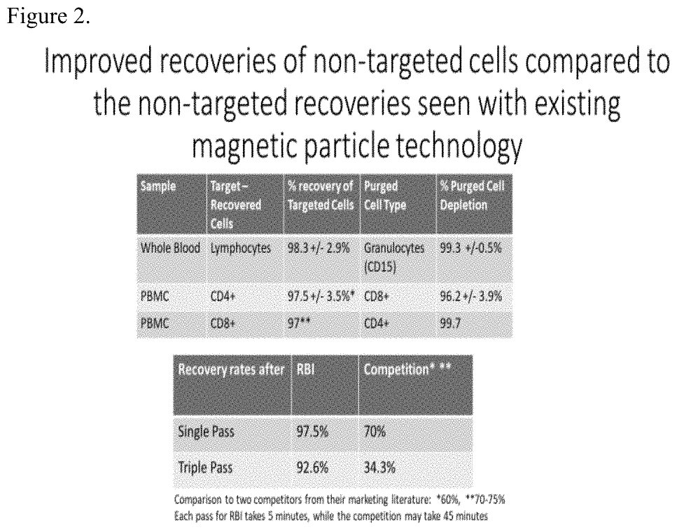

A key feature of the invention is the ability to remove the undesired cell population by purging/depletion using particles of the invention without loss of cells required for the cell therapeutic i.e. CAR T cells, but not limited to. The ability to remove an undesired cell population often will require multiple rounds of purging/depletion. As shown in FIG. 1, even purging CD8 cells for six rounds CD4 cells are not lost. FIG. 2 further demonstrates this unique feature of the particles of the invention as compared to other particles of the art.

The particles of the invention are composed of solid metal. As a result of this sterilization of the particles, an absolute requirement for a therapeutic application, is straight forward compared to magnetic particles of the art composed of iron oxides. The particles, prior to the addition of sterile reactants to the metal surface, are simply heated to 250 degrees centigrade for at least 3 hours to sterilize the particles which includes destroying endotoxin. The sterile particles are bound with sterile reactants by procedures described herein under sterile conditions to produce the sterile product.

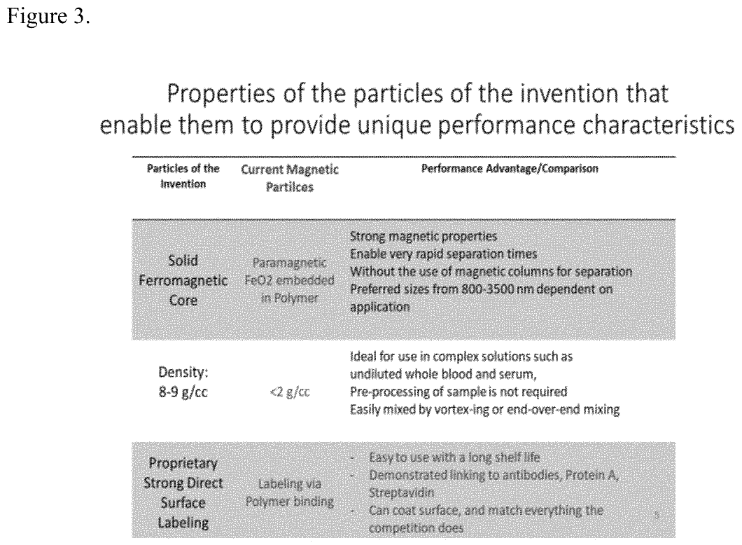

The particles of the invention have certain properties as detailed below that include being magnetic and having a density range and size (diameter) range. The particle is distinguishable from particles of the art as seen in FIG. 3 and U.S. Pat. No. 9,739,768 (′768) incorporated herein by reference. Key features of the particle as detailed in FIG. 3 are: its ferromagnetic core that results in the particle remaining magnetic upon removal of a magnetic field; the particle being composed of magnetic metals such as iron, nickel, cobalt and alloys thereof; the density of the particle being at least 3 times the density of cells with a preferred density for nickel about 8-9 g/cc. The particle can range in size/diameter from 500 nanometers to 5,000 nanometers.

The preferred particle in the preferred embodiment is composed of nickel with an oxide coating obtained by heating the particles to 250 degree centigrade for 3-24 hours. Other magnetic particles that will operate in the invention include any magnetic metal, metal oxide or alloys including those of nickel, iron, cobalt but not limited to. As long as the particle is composed of magnetic metal or magnetic metal/metal oxide or metal alloy and the particle remains magnetic when the magnetic field is removed is anticipated by the invention.

Particles of the invention are magnetically separated from biological fluids such as apheresis material (leukapheresis); Peripheral Blood Mononuclear Cells (PBMCs) and whole blood (diluted or undiluted). The invention also anticipates any biological fluid other than those listed in the previous sentence that may be used in the manufacture of products to be used for cell-based therapies. The magnetic particles are separated by an external magnetic field. The external magnetic field is typically applied by another permanent magnet or electromagnet. Permanent magnets such as those provided by Dexter magnetics or BD Biosciences, but not limited to, operate in the disclosed invention. Any magnet that moves the magnetic particles from the solution to the wall of the vessel containing the biological fluid is covered by the invention. The particles can be magnetically pulled to a point in the vessel or can be spread over the entire surface of the vessel or some part thereof as detailed in U.S. Pat. No. 5,466,574 (574). It will be obvious to one skilled in the art that as the particle size decreases a stronger magnetic field may be required to accomplish the required magnetic separation than that for a larger diameter particle. Such experimentation is anticipated by the disclosed invention. For magnetic separation in a 450 ml blood bag the magnetic separation can be performed by placing the blood bag on a plate magnet as an example only. As with conical test tubes, any magnetic configuration that removes the particles from the biological sample fluid so that the biological fluid can be removed from the magnetic particles is anticipated by the invention.

In order for the magnetic particle to remove an undesired cell population a reactant is bound to the magnetic particle that recognizes a cell surface antigen present on the undesired cell population. A reactant is any molecule that satisfies this requirement. Reactants include but are not limited to: monoclonal or polyclonal antibodies to cell surface antigens; lectins that bind to carbohydrate molecules on the cells or interest; biotin/avidin(streptavidin) where avidin (streptavidin) recognize molecules bound to the cell surface of interest that have a biotin moiety bound thereto. The reactant is bound to the surface of the dense, magnetic particle by means known in the art that include adsorption and covalent coupling. For adsorption the nickel particle is simply mixed with an appropriate reactant (for antibodies at approximately 2 mg antibody/meter squared) such as mouse antihuman monoclonal antibody overnight, rinsed, blocked with BSA and used to remove cell populations present in the biological fluid. All isotypes work by adsorption with IgM forming the most stable complex. Both adsorption and covalent coupling are detailed in US patent '799 and U.S. Pat. No. 9,739,768 (768) which are incorporated here by reference. It is clear that one skilled in the art will determine the best amount of reactant to bind to the particle for a given reactant by experimentation, such experimentation being anticipated by the invention disclosed herein.

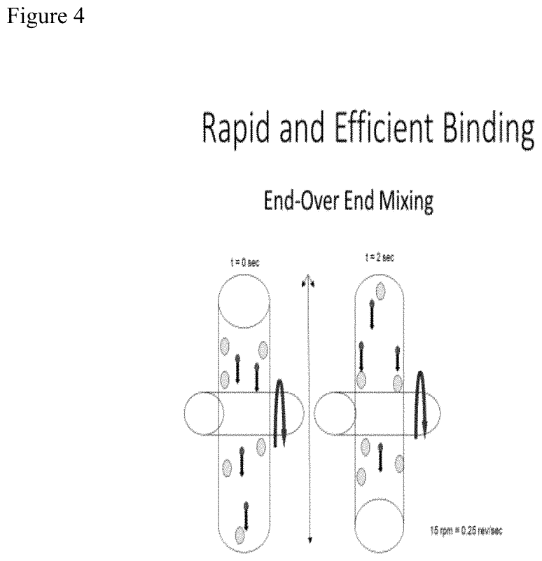

A major distinction from the use of magnetic separation technology between research applications and therapeutic applications is sample volume. For use in the invention described herein it is anticipated that sample volumes will be in the 20-500 ml range but not limited to. A key feature of the particles of the invention is their density. For the preferred embodiment the density of nickel is 8.9 g/cc. The density difference between cells and magnetic particles results in very convenient mixing. Due to the density differences between cells and the particles of the invention mixing causes the particles to traverse gently past the target population or subpopulation and bind efficiently to recognition sites with little or no non-specific binding to non-targeted populations or subpopulations of cells. Any mixing process must promote an effective movement of particles past their target population. By mixing the sample solution with magnetic particles therein in an end-over-end fashion as described in '799 binding occurs (FIG. 4). End-over-end mixing is conveniently accomplished by a variable speed mixer such as that provided by ATR Biotech. It is also envisioned that the invention will operate in a blood bag of approximately 450 ml. Any apparatus for mixing a blood bag that leads to mixing as required by the invention is anticipated by the invention disclosed here. For each application one skilled in the art will realize the need to optimize rotation speed for a given magnetic particle at a specific size. For the preferred embodiment using a 0.8 micron nickel particle the mixing speed is around 15-30 rpm. Any mixing process that promotes movement of the particles relative to a target population falls within the scope of the invention.

Nickel particles are commercially available from suppliers i.e. Sigma Aldrich and Novamet but a preferred particle is that described in '799.

The method for the preferred embodiment requires a nickel particle with an oxide coating with the appropriate reactant bound thereto. A sample biological fluid i.e. pheresis material, PBMCs, whole blood, but not limited to is added to the particles or the particles can be added to the biological fluid. Particles are removed from stock solutions, rinsed as described below and rinse buffer is removed. The sample biological fluid can then be added directly to the particle pellet at the bottom of the tube. The sample is vortexed briefly and mixed by end-over-end mixing for the appropriate time and then the tube is placed in a magnetic field for the appropriate time and the sample is removed and used in the next step required to create i.e. CAR T cells for treatment of a cancer patient. It is anticipated though not required that when a sterile blood bag is used the particles with the appropriate reactant bound thereto may already be in the blood bag or be added by a sterile procedure through a port in the blood bag.

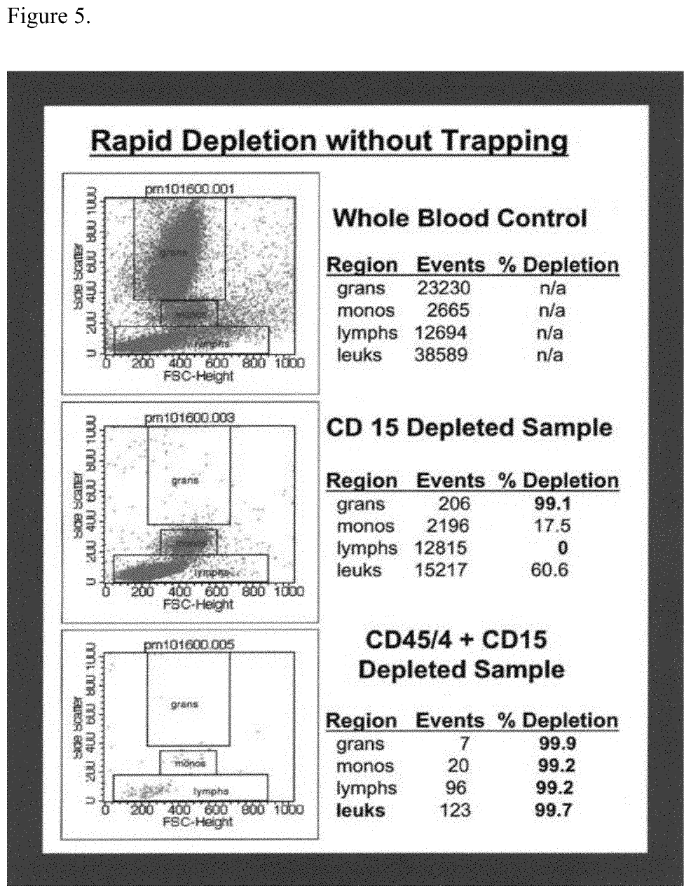

Due to the density differences between cells and the particles in the practice of the invention mixing causes the particles to traverse gently past the target population or subpopulation and bind efficiently to recognition sites with little or no non-specific binding to non-targeted population or subpopulations of cells FIGS. 1 and 5. Any mixing process must promote an effective movement of particles past their target population.

Details of the protocol are shown below for the removal of CD15+ granulocytes from whole blood (FIG. 5). It is to be understood that one skilled in the art can vary the protocol as needed to determine the best particle to use, the best mixing time and magnetic separation time for each nickel particle/reactant chosen.

IMPORTANT: DO NOT ALLOW PARTICLES TO COME IN CONTACT WITH A MAGNETIC FIELD UNTIL DIRECTED TO DO SO IN THE PROTOCOL.

Method:
1. Add desired CD15 magnetic particles (50 ul/ml whole blood, PBMCs or apheresis material to be processed*) to appropriate volume of rinse buffer determined by experimentation.
2. Rinse particles (See rinsing procedures below) using rinse buffer.
3. Remove rinse buffer from final rinse and add sample (whole blood, PBMC or apheresis material) and vortex for a few seconds
4. Mix on end-over-end mixer for 5-30 minutes*
5. Place tube in magnetic field for 1-5 minute*
6. Transfer sample to a clean sterile container for further processing in the manufacture of the desired cell population for use as a cell based therapeutic. * The actual amount of particles/ml, mixing time and/or magnetic separation time should be varied to determine the best parameters for the cell type/monoclonal antibody(s)/reactants used in the process for the preparation of i.e. CAR T cells but not limited to CAR T cells.

Rinsing Procedures

Magnetic particles can be rinsed in three different ways: 1. centrifugation, 2. gravity settling or 3. magnetic field exposure. Procedure 1 or 2 is recommended. If rinsing using magnetic separation, particles should be demagnetized prior to use as described below.

Rinsing Buffer is sterile PBS/0.1% BSA/pH 7.2.

Final resuspension buffer can be Rinse Buffer or PBS.
1. Centrifugation: Vortex bottle well before removing desired quantity of magnetic particles. Centrifuge at 500 rpm for 30 seconds, remove buffer; add fresh buffer; resuspend particles by vortexing or pipette up and down. Repeat one time. Resuspend to original volume of Rinse Buffer
2. Gravity Settling (Preferred rinsing procedure): Vortex bottle well before removing desired quantity of magnetic particles. Allow particles to settle by gravity for 2-3 minutes; remove buffer; add fresh buffer; resuspend particles by vortexing or pipette up and down. Repeat one time. Resuspend to original volume of Rinse Buffer.
3. Magnetic Separation: Vortex bottle well before removing desired quantity of magnetic particles. Place particles in magnetic separator for 4-5 seconds; remove buffer; remove test tube from magnetic separator; add buffer; resuspend by vortexing or pipette up and down. Repeat one time. Resuspend to original volume of Rinse Buffer. Demagnetize the final particle suspension (see demagnetizer under Equipment)

Equipment Required for Optimal Performance of CD15 Magnetic Particles:
1. Mixer: Due to the difference in density between magnetic particles and cells proper mixing is essential to ensure contact between the particles and the targeted cells. The bead cell mixture cannot be vortexed briefly and allowed to stand without mixing. For volumes >2 mL mixing can be accomplished by end-over-end mixing using an ATR Rotomix mixer (www.atrbiotech.com/benchtop/rotomix.htm) with variable speed. Recommended mixing speed is 15-30 rpm.
2. Magnetic Separation:
   Magnets for use with magnetic particles can be obtained from Dexter Magnetic Technologies (www.lifesep.com under products). Different magnets are available for sample volumes from <0.5 mL to 50 mL. For use in a blood bag appropriate magnets will be used that effectively collect the particles so that the biological sample can be removed without carryover of the magnetic particles.
3. Demagnetizer: To ensure that particles are well dispersed, it is recommended, but not required unless particles are exposed to a magnetic field prior to use, that the particles be demagnetized immediately prior to the addition to the sample, using a demagnetizer/degausser from Data Devices International (http://www.datadev.com/degausser-small-office-data-security.html). Model PF211.

Hold the demagnetizer at the bottom of the tube containing the particles; (hold test tube in one hand; demagnetizer in the other hand; demagnetizer can touch test tube); turn on by holding the “on button” in the on position; rotate demagnetizer in a clockwise or counterclockwise motion for 10-12 seconds; WHILE UNIT IS STILL ON (if unit is turned off before this step particles will be magnetized) slowly move the demagnetizer away from the test tube (about 3 feet; arm's length); turn off

The appropriate number of beads required to remove the undesired cell population(s) by the invention disclosed herein will be determined experimentally by varying bead size, mixing speed and magnetic separation times for each application.

Cell therapy manufacturers want as few granulocytes, platelets, and RBCs contamination as possible in the final therapeutic product that is used to treat cancer. The invention disclosed herein will be effectively used to remove these cell populations by manufacturing particles with the appropriate reactants bound there to and performing the method as disclosed herein by sterile procedures. The removal of platelets is further detailed in WO/2018/231373 incorporated herein by reference.

Often it is desired to not only purge cell populations but following a purging step to add a step to further purify the cells by positive selection that is accomplished by positive magnetic particle separation (Miltenyi or ThermoFisher but not limited to) or Fluorescent Activated Cell Sorting (FACS). Because of the very high recovery of desired cells seen with the invention disclosed herein it would be obvious for one skilled in the art to use the invention disclosed here in combination with positive cell selection technology.

The use of the preferred embodiment of the invention to rapidly deplete cell populations and yield a desired cell population in high yield for the manufacturing process for producing cell-based therapies will be described in the following examples, which are intended to be illustrative of the invention, but in no way limiting of its scope.

### Example 1

In order to have a robust manufacturing procedure for autologous CAR T cell therapy for B cell Lymphoma it is necessary that following apheresis that the apheresis product be depleted of any B-cell cancer cells. The best way to accomplish this is using magnetic separation technology to remove any B cell cancer cells and also following the depletion step to leave the desired cells at close to 100% recovery as possible. Patients undergoing such treatment are very sick and the loss of desired cells for the production of CAR T cells is unacceptable. The method disclosed in this invention yields desired cells following a purging/depletion step in acceptable yields. Because the invention results in such high recovery of desired cells it is possible that these very sick patients may have to undergo fewer apheresis procedures.

To remove possible contaminating B-cell cancer cells from the apheresis material obtained from patients, the method disclosed here will be performed using particles of the invention with the following reactant(s) bound thereto. The following reactants, anti-CD19 and/or CD20, will be bound to the particles by adsorption or by covalent coupling by means known in the art. The particles will be manufactured sterilely as described herein. Any other reactant that recognizes antigens on the B cell cancer cells is anticipated by the invention.

All procedures will be carried out under sterile conditions. Apheresis material will be used directly or following Ficoll centrifugation of the apheresis material. The particles will be rinsed and placed in the bottom of a 50 ml conical centrifuge tube. The cell material will be centrifuged by means known in the art. The supernatant will be removed down to a volume of 45-50 ml. The cells will be re-suspended and added to the magnetic particle pellet and resuspended as described by the method disclosed herein. The solution will be mixed by end-over-end mixing at 15-30 rpm as necessary to bind to the B-cell cancer cells. The optimal mixing time will be determined by routine experimentation. Following mixing the 50 ml conical centrifuge tube will be placed in a magnetic field for sufficient time (to be determined by routine experimentation). The supernatant devoid of cancer cells will be removed form the conical centrifuge tube and used for the next step in the CAR-T cell production procedure. The method will be repeated until cancer cells are not present as determined by methods known in the art such as PCR. Recovery of desired cells required for the production of CAR T cells will be determined usually by Flow Cytometric analysis.

It is anticipated that ultimately the goal is to perform the magnetic depletion step in a sterile blood bag with a volume up to 450 ml but not limited to. The procedure will work in a blood bag following appropriate experimentation for mixing time, magnetic separation time and number of particles as long as the blood bag can be rotated such that the particles mix as required by the invention disclosed herein.

### Example 2

In the case of Car T cell treatment for B cell Lymphoma it is extremely important even after the treatment discussed in Example 1 to treat the final CAR T cell preparation before infusion into the patient to further ensure that no cancer cells are infused into the patient. This is only possible with the invention disclosed herein because of the high recovery of non-targeted cells in this case the CAR T cells that will be used to treat the patient. Possible residual B cell cancer cells will be purged as detailed in Example 1.

### Example 3

It is clear that investigators studying both autologous and allogeneic cell therapies for the treatment of cancer are moving away from using PBMCs for i.e. CART cell preparation in favor of using certain lymphocyte subsets such as CD4 T cells and CD8 T cells. It is also clear that at the time of this disclosure it is not clear what is required i.e. what ratio of CD4 T cells/CD8 T cells. Once this question is answered the invention disclosed here will be used by one skilled in the art to determine the appropriate reactants to bind to the particles to obtain the desired cells to further the development of the CAR T cells for therapeutic application.

While the present invention has been described in terms of its preferred embodiment, it is to be appreciated that the invention is not limited thereby, and that one skilled in the art can conceive of numerous variations and modifications of the invention as described herein, without departing from the spirit and scope of the following claims.

### CITED REFERENCES

### Patents/Patent Applications

| | | |
|---|---|---|
| 5,576,185 | Coulter | Nov. 19, 1996 |
| 9,435,799 | Russell | Sep. 6, 2016 |
| 5,466,574 | Liberti | Nov. 4, 1995 |
| 5,411,863 | Miltenyi | May 2, 1995 |
| 4,654,267 | Ugelstad | Mar. 31, 1987 |
| 9,739,768 | Russell | Aug. 22, 2017 |
| 4,707,523 | Chang | Nov. 17, 1987 |
| 10,117,896 | Powell | Nov. 6, 2018 |
| 20180256712 | June | Sep. 13, 2018 |
| 10,093,900 | Jantz | Oct. 9, 2018 |
| 10,081,793 | Kokaji | Sep. 25, 2018 |
| WO/2018/231373 | Russell | Dec. 21, 2018 |

1. Tricket, Annette; T Cell Stimulation and Expansion Using Anti-CD3/CD28 Beads; J. Immunological Methods; Vol 275 Page 251; (2003).
2. Kohl, Ulrike, et al; CAR T Cells in Trials: Recent Achievements and Challenges that Remain the production of Modified T Cells for Clinical Applications; Human Gene Therapy; Vol 29 Page 559 (2018)
3. Iyer, Rohin et al; Industrializing Autologous Adoptive Immunotherapies: Manufacturing Advances and Challenges; Frontiers in Medicine; Vol 5 Page 1 (2018)
4. Wang, Xiuyan and Riviere, Isabelle; Clinical Manufacturing of CAR T Cells: Foundation of a promising Therapy; Molecular Therapy—Oncolytics; Vol 3 Page 1 (2016)
5. Themeli, Maria, et al; New Cell Sources for T Cell Engineering and Adoptive Immunotherapy; Cell Stem Cell; Vol 16 P 2, age 357 (2015)
6. Pagliaro, Ned; www.biopharmadive.com/news; published Oct. 2, (2018)
7. CAR T Cell Therapies and CRISPR are fighting Cancer and Revolutionizing Medicine; www.synthego.com.blog/car-t-crispr-cancer; posted May 8, (2018)
8. Tutle, C J et al; CD19 CAR T Cells of Defined CD4+:CD8+J, Clin Invest; Vol 126 Page 2123 (2016)
9. Summermeyer, D et al; Chimeric antigen receptor-modified T cells derived from defined CD4+ and CD8+ subsets confer superior antitumor reactivity in vivo; Leukemia; Vol 30 Page 492 (2016)
10. Willinger, Tim et al; Molecular Signatures Distinguish Human Central Memory from Effector Memory CD8 T Cell Subsets; J. Immunology; Vol 175 Page 5895 (2005)
11. Friedberg, J W et al; Ex vivo B cell depletion using ELIGIX B Cell SC system and autologous peripheral blood stem cell transplantation in patients with follicular non-Hodgkin's lymphoma; Bone Marrow Transplantation; Vol 32 Page 681 (2003)
12. Alyea, E P et al; CD8+ cell depletion of donor lymphocyte infusions using CD8+ monoclonal antibody-coated high-density microparticles after allogeneic hematopoietic stem cell transplantation” a pilot study; Bone Marrow Transplantation; Vol 34 Page 123 (2004)
13. Bersenov, A; New Method for Cord Blood Processing and cd34+ Cell Separation; http://Stemcellassays.com/2013/02/method-cord-blood-processing-cd34-cell-separation/
14. Collins, R H et al.; Donor leukocyte infusions in 140 patients with relapsed malignancy after allogeneic bone marrow transplantation; J. Clin. Oncol. Vol 15 Page 433 (1997)
15. Joseph, A; In rare case, patient developed resistance to CAR-T after cancer cell began hiding in ‘plain sight’; www.statnews.com/2018/10/01/cae-t-resistance-cancer-cell-hiding/
16. Nawrat, A; patient death following CAR-T therapy highlights serious manufacturing issues; www.pharmaceutical-technology.com/digital-disruption/patient-death-following-car-t-manufacturing
17. Bell, J.; 3 Questions leaving ASH (2018); www.biopharmadive.com/new/3-questions-leaving-ash/543686/
18. Bersenev, A. Crude versus defined CAR-T cell therapy product. Cells Weekly May 1^{st} (2016)
19. MacLeod, T C et al; Integration of a CD19 CAR into the TCR Alpha Chain Locus Streamlines Production of Allogeneic Gene-Edited CAR T cells. Molecular Therapy. Vol 4; page 949 (2017)
20. https://labiotech.eu/features/gamma-delta-t-cells-cancer/
21. Brown, C E et al: Regression of Glioblastoma after Chimeric Antigen Receptor T-Cell Therapy. New England Journal of Medicine; V 375; page 2561 (2016).

I claim:

1. A method for improving the preparation of cell-based therapies in treating a patient with cancer comprising:
a. obtaining a volume of a sample of biological fluid in a vessel from the patient;
b. enriching desired cells using magnetic, dense metal particle selection bound to reactants; and
c. recovering greater than 80% of desired cells wherein the desired cells are used in cell-based therapies wherein the improved preparation is manufactured commercially.

2. The method of claim 1 where the sample of biological fluid is apheresis material.

3. The method of claim 1 where the sample of biological fluid is selected from a group consisting of peripheral blood mononuclear cells, diluted whole blood, and undiluted whole blood.

4. The method of claim 1 where the magnetic, dense metal particle selection includes application of an external field from a permanent magnetic or electromagnet.

5. The method of claim 1 where the magnetic, dense metal particle selection includes magnetically pulling the particles to a point in the vessel or spread over a portion of a surface of the vessel.

6. The method of claim 1 where the vessel is a 450 ml blood bag.

7. The method of claim 1 where the volume is between approximately 20 to 500 ml.

8. The method of claim 1 where the magnetic, dense metal particle selection includes end-over-end mixing.

9. The method of claim 1 where the magnetic, dense metal particles are selected from a group consisting of iron, nickel, cobalt and alloys thereof.

10. The method of claim 1 where the magnetic, dense metal particles have a density at least 3 times a density of the undesired cells.

11. The method of claim 9 where the magnetic, dense nickel particles have a density of about 8 to 9 g/cc.

12. The method of claim 1 where the magnetic, dense metal particles have a size of approximately 500 to 5000 nm.

13. The method of claim 1 where the magnetic, dense metal particles are nickel particles with an oxide coating obtained after heating to 250 degrees centigrade for 3 to 24 hours.

14. The method of claim 1 where the reactants are from a group consisting of monoclonal antibodies, polyclonal antibodies lectins, and streptavidin.

15. The method of claim 1 where enriching desired cells is by removing undesired cells.

16. The method of claim 15 where the reactants are anti-CD8.

17. The method of claim 1 where the reactants are anti-CD15.

18. The method of claim 1 where enriching desired cells is by selecting desired cells.

19. The method of claim 18 where the reactants are anti-CD4.

20. The method of claim 15 where the undesired cells are B-cell cancer cells.

21. The method of claim 20 where the reactants are anti-CD19 or anti-CD20.

22. The method of claim 1 where the cell-based therapy involved the preparation of CAR T cells.

23. The method of claim 22 where the CAR T cells are used in autologous or allogeneric CAR T cell therapy.

24. The method of claim 1 where the magnetic, dense nickel particles are sterilized by heating to 250 degrees centigrade for an appropriate time.

25. The method of claim 1 wherein the recovery of undesired cells for the production of CAR T cells is confirmed by Flow Cytometric Analysis.
